(19) United States
(12) Patent Application Publication
(10) Pub. No.: US 2016/0166602 A1
(43) Pub. Date: Jun. 16, 2016

(54) Food Supplement Composition

(71) Applicant: Homeland Housewares, LLC, Los Angeles, CA (US)

(72) Inventor: Lenny Sands, Los Angeles, CA (US)

(73) Assignee: HOMELAND HOUSEWARES, LLC, Los Angeles, CA (US)

(21) Appl. No.: 14/569,555

(22) Filed: Dec. 12, 2014

(51) Int. Cl. A61K 31/733; A61K 36/537; A61K 36/82; A61K 36/54; A61K 36/906; A61K 36/9066; A61K 36/81; A61K 36/258; A61K 36/53; A61K 36/41; A61K 36/31; A61K 35/748; A61K 36/899; A61K 36/48; A61K 36/05; A61K 36/87; A61K 36/73; A61K 36/61; A61K 36/07; A61K 36/88; A61K 36/67; A61K 36/064; A23L 1/30; A61K 36/185

(52) U.S. Cl. CPC A61K 31/733; A61K 36/185; A61K 36/537; A61K 36/82; A61K 36/54; A61K 36/906; A61K 36/9066; A61K 36/81; A61K 36/258; A61K 36/53; A61K 36/41; A61K 36/31; A61K 35/748; A61K 36/899; A61K 36/48; A61K 36/05; A61K 36/87; A61K 36/73; A61K 36/61; A61K 36/07; A61K 36/88; A61K 36/67; A61K 36/064; A23L 1/3002; A23L 1/3016; A23L 1/30; A23V 2002/00

(57) ABSTRACT

A dietary supplement for promoting increased vitality, supporting mood, improving heart health, improving immune strength, improving antioxidant balance, protecting against free radical damage, improving bowel function, and enhancing flavonoid intake. The composition is a combination of cacao, chia seeds, green tea, cinnamon, ginger and turmeric in specific percents of the formulation. Other compositions include constituents of inulin, Ashwagandha, green tea, ginseng, Bacopa monnieri, Rhodiola and maca root. Still other compositions include a combination of Spirulina, wheat grass, alfalfa, chlorella and cinnamon powder. Other compositions include constituents of inulin, acerola, grape, beet, current, acai, raspberry, blackberry, pomegranate, lingonberry, mangosteen, and blueberry. Other compositions include maitake mushroom, purple corn, turmeric, cane juice, onion, basil, black pepper, coriander, oregano, rosemary, thyme, parsley, celery seed, bay leaf, tomato, garlic, yeast, salt, carrot, and spinach.

### CROSS REFERENCE TO RELATED APPLICATIONS

None.

### FIELD OF THE INVENTION

The invention relates to a food mixture or a category of food mixtures commonly known as superfoods. In particular, the invention relates to organic, natural foods used to supplement the diet for use in vegetable and fruit-based beverages.

### BACKGROUND OF THE INVENTION

The term “superfood” is used in the dietary supplement industry to describe foods that impart health benefits, alleviate medical conditions, and are beneficial for general health and well-being. People are often looking for simple ways to introduce superfoods into their diet. In addition, many individuals would like foods that are tasty, organic, and gluten-free that can be consumed alone or added to food and drink.

Dietary supplement compositions have been used to treat various ailments and produce general improved health. In U.S. Pat. No. 5,925,377, Gerth describes a supplement using a combination of amino acids, minerals, vitamins, and herbs to synergistically act as an appetite suppressant and convert stored fat into energy. In U.S. Pat. No. 7,923,044, Bias describes a dietary supplement that uses powdered grass, algae, freeze-dried vegetables, oat bran, cinnamon and other naturally occurring substances, for reducing cellular damage and preventing premature aging caused by free-radicals. In U.S. Pat. No. 8,257,694, Daikeler describes a nutritional composition for reducing oxidative damage by a composition that includes superfoods such as maca root, Goji berries, mushrooms, basil, and other naturally occurring substances. However, there still remains a need for natural supplements that improve health and well-being. The contents of all references throughout this application are incorporated by reference in their entireties, for all purposes.

### SUMMARY OF THE INVENTION

The nutritional compositions disclosed herein have been shown to have one or more of the following benefits: increase vitality, increase energy, support mood, improve heart health, support immune strength, maintain antioxidant balance, maintain pH balance, support normal bowl function, maintain healthy circulation, protect against free radical damage, improve plasma glucose levels, enhance flavonoid intake, and support healthy joint function.

A general objective of the present invention is to provide highly nutritious, organic groups of foods that can easily be added to food or drinks. In particular, a purpose of the embodiments of the invention is to provide an organic, gluten-free food that can be consumed alone or added to foods and drinks to provide antioxidants, flavonoids, phytochemical, to increase the energy and vitality of an individual in an easy to use delivery system.

Another objective of the present invention is to provide a dietary supplemental composition, which can be readily mixed with or dissolved in other foods (sprinkled on yogurt, mixed with other beverages, added to cereals, etc.). A further objective of the present invention is to provide a 100% vegetarian composition, with no ingredients that are commonly associated with allergens, without added sweeteners, and without artificial flavors or any excipients or fillers.

The embodiments of the invention are a blend of organic superfoods in whole foods or ground powder. Some embodiments can easily be added to existing foods or beverages. Some constituents of the compositions in the embodiments of the invention are made using unique processes of cleaning and drying that retain the nutrient profile of the raw unprocessed constituents.

In accordance with an embodiment, the nutritional composition comprises the constituents of: cacao, chia seed, green tea, cinnamon, ginger, and turmeric. In a particular embodiment, the cacao is in the form of cacao powder, the chia seed is in the from of whole chia seed, the green tea is in the form of green tea powder, the cinnamon is in the form of cinnamon powder, the ginger is in the form of ginger powder, and the turmeric is in the form of turmeric powder.

In accordance with yet another embodiment, the nutritional composition comprises the constituents of: inulin, Ashwagandha, green tea extract, Panax ginseng, Bacopa monnieri, Rhodiola extract, maca root and vanilla.

In accordance with yet another embodiment, the nutritional composition comprises the constituents of: Spirulina (Spirulina platensis), wheat grass (Triticum aestivum L), alfalfa, chlorella (Chlorella pyrenoidosa or Chlorella vulgaris), cinnamon, and vanilla. In a preferred embodiment, the Spirulina is in the form of powder, the wheat grass is in the form of powder from young leaves, the alfalfa is in the form of a whole grass powder from young leaves, and the cinnamon is in the form of powder.

In accordance with yet another embodiment, the nutritional composition comprises a formulation of: inulin, acerola, concord grapes, beets, black current, acai, raspberry extract, pomegranate extract, lingonberry extract, mangosteen, and blueberry extract. In a preferred embodiment, the acerola is in the form of acerola extract, the beets are derived from beet root, the black currant is in the form of a 20:1 extract, the raspberry is in the form of a raspberry extract 20:1, the pomegranate is in the form of pomegranate extract 20:1, the lingonberry is in the form of lingonberry extract 20:1, and the blueberry is in the form of blueberry extract 25:1.

In accordance with yet another embodiment, the nutritional composition comprises a formulation of of: maitake mushroom, purple corn, turmeric, cane juice, opinion, basil, black pepper, coriander, oregano, rosemary, thyme, parsley, celery seed, bay leaf, tomato, garlic, yeast, salt, carrot, and spinach. In a particular embodiment, the maitake mushroom is in the form of a powder, the purple corn is in the form of purple corn powder, the turmeric is in the form of powder, the cane juice is in the form of evaporated cane juice, and the salt is Himalayan salt.

In accordance with yet another embodiment, a method of beneficially affecting any of the health benefits described above is produced by orally administering any one or more of the compositions to produce the health benefit.

Preferably, all constituents in the above formulations are organic. In any of embodiments, the composition may be comprised of, consist of, or consist essentially of some or all of the listed constituents. Other objects, features, and advantages of the present invention will become apparent to those skilled in the art from the following detailed description.

### DETAILED DESCRIPTION OF THE EMBODIMENTS

A more complete appreciation of the disclosure and many of the associated advantages will be readily obtained, as the same becomes better understood by reference to the following detailed description of the exemplary embodiments.

In a preferred embodiment that increases the body's ability to burn fat, the nutritional composition comprises at least one, but preferably all, of the constituents in Table 1. The amounts provided preferably have a serving size of approximately 5 grams. A single service has approximately 20 calories, 3 grams of total carbohydrate, of which 2 grams are dietary fiber, and 1 gram of protein. The percent of each of the formulations listed below is by percent weight.

TABLE 1

| | Preferred | |
|---|---|---|
| Constituent | Percent Range | Preferred Percent |
| Organic Cacao Powder | 23.0%-43.0% | 33.0% |
| Organic Whole Chia Seed | 17.0%-37.0% | 27.0% |
| Organic Green Tea Powder | 4.0%-24.0% | 14.0% |
| Organic Cinnamon Powder | 5.0%-25.0% | 15.0% |
| Organic Ginger Powder | 1.0%-21.0% | 11.0% |
| Organic Turmeric Powder | 0.1%-15.0% | 5.0% |

In a preferred embodiment to increase energy, the nutritional composition comprises at least one, but preferably all, of the constituents in Table 2. The amounts provided preferably have a serving size of approximately 5 grams. A single service has approximately 20 calories, 3 grams of total carbohydrate, of which 2 grams are dietary fiber, and 2 grams of protein.

TABLE 2

| | Preferred | |
|---|---|---|
| Constituent | Percent Range | Preferred Percent |
| Organic Inulin | 62.4%-82.4% | 72.4% |
| Organic Ashwagandha | 1.0%-16.0% | 6.0% |
| (Withania somnifera (KSM- | | |
| 66)) | | |
| Organic Green Tea Extract | 0.1%-17.6% | 7.6% |
| Powder (Std. to 20% | | |
| caffeine) | | |
| Organic Panex Ginseng | 0.1%-0.8% | 0.4% |
| (Asian ginseng - Araliaceae) | | |
| Organic Bacopa monnieri | 0.1%-18.6% | 8.6% |
| (leaf) | | |
| Organic Rhodiola extract | 0.1%-4.0% | 2.0% |
| (Rhodiola rosea); 1% | | |
| salidrosides | | |
| Organic Maca root | 0.1%-2.0% | 1.0% |
| Organic Vanilla flavor | 0.1%-4.0% | 2.0% |

In a preferred embodiment to increase the body's ability to detoxify, support immune strength, maintain antioxidant and pH balance, support normal bowel function, and maintain healthy circulation, the nutritional composition comprises at least one, but preferably all, of the constituents in Table 3. The amounts provided preferably have a serving size of approximately 5 grams. A single serving has approximately 30 calories, 15 calories from fat, less than one gram of total fat, 2 grams of total carbohydrate of which 1 gram is dietary fiber, 1 gram of protein and approximately 4250 IU of Vitamin A.

TABLE 3

| | Preferred | |
|---|---|---|
| Constituent | Percent Range | Preferred Percent |
| Organic Spirulina | 20.0%-40.0% | 30.0% |
| (Spirulina platensis) | | |
| whole plant (algae) | | |
| Organic Wheat Grass Powder | 20.0%-40.0% | 30.0% |
| (Triticum aestivum L) from | | |
| young leaves | | |
| Organic Alfalfa Whole Grass | 17.0%-37.0% | 27.0% |
| Powder (Medicago sativa) | | |
| from young leaves | | |
| Organic Chlorella | 0.1%-18.0% | 8.0% |
| (Chlorella pyrenoidosa | | |
| or Chlorella vulgaris) | | |
| Organic Cinnamon Powder | 0.1%-6.0% | 3.0% |

In a preferred embodiment to protect against free radical damage, support healthy joint function, provide 100% RDA of vitamin C, the nutritional composition comprises at least one, but preferably all, of the constituents in Table 4. The amounts provided preferably have a serving size of approximately 5 grams. A single serving has approximately 20 total calories, 5 calories from fat, less than one gram of total fat, 3.7 grams of total carbohydrate of which less than 1 gram is dietary fiber and approximately 4 grams are from dietary fiber, and 1.6 mg of iron.

TABLE 4

| | Preferred | |
|---|---|---|
| Constituent | Percent Range | Preferred Percent |
| Organic Inulin | 65.6%-75.6% | 70.6% |
| Organic Acerola (fruit) | 2.0%-12.0% | 7.0% |
| Extract (NMT 17% Vitamin | | |
| C) | | |
| Organic Concorde Grape | 0.1%-6.0% | 3.0% |
| (whole) | | |
| Organic Beet (root) | 0.1%-6.0% | 3.0% |
| Organic Black Currant | 0.1%-4.0% | 2.0% |
| Extract 20:1 | | |
| Organic Acai (fruit) | 0.1%-4.0% | 2.0% |
| Organic Raspberry Extract | 0.1%-3.0% | 1.5% |
| 20:1 | | |
| Organic Pomegranate Extract | 0.1%-3.0% | 1.5% |
| 20:1 | | |
| Organic Lingonberry Extract | 0.1%-3.0% | 1.5% |
| 20:1 | | |
| Organic Mangosteen (whole) | 0.1%-3.0% | 1.5% |
| Organic Blueberry Extract | 0.1%-2.0% | 1.0% |
| 25:1 | | |
| Other constituents: natural | 0.4%-10.4% | 5.4% |
| wild berry flavors, citric acid | | |
| (150 mg), organic stevia | | |

In a preferred embodiment to protect against free radical damage, support healthy joint function, provide 100% RDA of vitamin C, the nutritional composition comprises at least one, but preferably all, of the constituents in Table 5. The amounts provided preferably have a serving size of approximately 5 grams. A single serving has approximately 20 total calories, 5 calories from fat, less than one gram of total fat, 3.7 grams of total carbohydrate of which less than 1 gram is dietary fiber and approximately 4 grams are from dietary fiber, and 1.6 mg of iron.

TABLE 5

| | Preferred | |
|---|---|---|
| Constituent | Percent Range | Preferred Percent |
| Organic Maitake Mushroom | 9.0%-29.0% | 19.0% |
| Powder | | |
| Organic Purple Corn Powder | 3.0%-23.0% | 13.0% |
| Organic Turmeric Powder | 0.1%-17.1% | 7.1% |
| Organic Evaporated Cane | 3.2%-23.2% | 13.2% |
| Juice | | |
| Organic Onion | 3.7%-23.7% | 13.7% |
| Basil | 0.1%-4.0% | 2.0% |
| Black Pepper | 0.1%-2.8% | 1.4% |
| Coriander | 0.1%-2.8% | 1.4% |
| Oregano | 0.1%-2.8% | 1.4% |
| Rosemary | 0.1%-2.8% | 1.4% |
| Thyme | 0.1%-2.8% | 1.4% |
| Parsley | 0.1%-0.8% | 0.4% |
| Celery Seed | 0.1%-0.8% | 0.4% |
| Bay Leaf | 0.1%-0.4% | 0.2% |
| Organic Tomato | 0.1%-19.3% | 9.3% |
| Organic Garlic | 0.1%-17.4% | 7.4% |
| Organic Yeast | 0.1%-7.0% | 3.5% |
| Salt (Himalayan) | 0.1%-6.0% | 3.0% |
| Organic Carrot | 0.1%-1.0% | 0.5% |
| Organic Spinach | 0.1%-1.0% | 0.5% |

Cacao has been known to support heart health, is rich in antioxidants, and supports bone strength. The cacao in the described compositions may be processed by traditionally used processing means, but the compositions have added benefits if the cacao is carefully sourced and processed. Organic, gentle cleaned cacao nibs retain phenolic compounds and flavonoids with antioxidant and mood enhancing properties. Roasting cacao beans, which is commonly practiced in the industry, reduces the amount of antioxidants and other nutrients in the cacao. To ensure that phenolic compounds are not compromised, the cacao beans are derived from freshly-split pods from good exporting countries, such as Peru, and are fermented under control temperatures (40° C.), washed and solar dried. The dried cacao beans are then cracked into nibs and the outer skin gently blown off with fans. The cacao nibs are placed into a hydraulic press that pressed out the oil of the cacao bean, which leaves behind a concentrated cacao cake that is low in fat (approximately 11% to 13% by weight, fat). The cacao nibs may be used in this state, or further cold milled into a powder, then sieved for use as cacao powder. This composition contains approximately 50% to 70% cacao. The cacao composition provides a significant amount of antioxidants and also imparts a familiar, satisfying flavor to users who add it to their food, drink or smoothie.

Maca is known to increase energy, improve performance, and increase vitality. In a preferred embodiment, the Maca root in the described compositions is selected from the Lepidium meyenii species that is grown in an altitude of approximately 9,000 to 14,000 feet (approximately 2700 to 4300 meters) above sea level.

Chia seeds are known to help maintain normal blood sugar levels, promote normal bowel function, and produce high alpha-linoleic and eicosapentaenoic acid levels, while not contributing to inflammation or disease risk factors.

The above description is for ease of reference only, and is not intended be a definitive description of the subject matter.

I claim:

1. A composition comprising:
about 23% to about 43% by weight cacao powder;
about 17% to about 37% by weight whole chia seed;
about 4% to about 24% by weight green tea powder;
about 5% to about 25% by weight cinnamon powder;
about 0.1% to about 15% by weight ginger powder; and,
about 0.1 to about 10.0% by weight turmeric powder.

2. A composition of claim 1 comprising:
about 33% by weight cacao powder;
about 27% by weight whole chia seed;
about 14% by weight green tea powder;
about 15% by weight cinnamon powder;
about 11% by weight ginger powder; and,
about 5% by weight turmeric powder.

3. A composition comprising:
about 62.4% to about 82.4% by weight inulin;
about 1% to about 16% by weight Ashwagandha;
about 0.1% to about 17% by weight green tea extract;
about 0.1% to about 0.4% by weight Panex ginseng;
about 0.1% to about 18.6% by weight Bacopa monnieri;
about 0.1% to about 4% by weight Rhodiola extract;
about 0.1% to about 2% by weight maca root; and
about 0.1% to about 4% vanilla flavoring.

4. A composition of claim 3 comprising:
about 72.4% by weight inulin;
about 6% by weight Ashwagandha;
about 7.6% by weight green tea extract;
about 0.4% by weight Panex ginseng;
about 8.6% by weight Bacopa monnieri;
about 2% by weight Rhodiola extract;
about 1% by weight maca root; and
about 2% vanilla flavoring.

5. A composition comprising:
about 20% to about 30% by weight Spirulina;
about 20% to about 40% by weight wheat grass powder;
about 17% to about 37% by weight alfalfa grass;
about 0.1% to about 18% by weight chlorella; and,
about 0.1% to about 6% by weight cinnamon powder;

6. The composition of claim 5 comprising:
about 30% by weight Spirulina;
about 30% by weight wheat grass powder;
about 27% by weight alfalfa grass;
about 8% by weight chlorella; and,
about 3% by weight cinnamon powder;

7. A composition comprising:
about 65.6% to about 75.6% by weight inulin;
about 2% to about 12% by weight acerola extract;
about 0.1% to about 6% by weight concord grape;
about 0.1% to about 6% by weight beet; and
about 0.1% to about 4.0% by weight black current extract having an extract ratio of about 20:1;
about 0.1% to about 4.0% by weight acai;
about 0.1% to about 3% by weight raspberry extract having an extract ratio of about 20:1;
about 0.1% to about 3% by weight pomegranate extract, having an extract ratio of about 20:1;
about 0.1% to about 3% by weight lingonberry extract having an extract ratio of about 20:1;
about 0.1% to about 3% by weight mangosteen;
about 0.1% to about 2% by weight blueberry extract having an extra ratio of about 25:1; and,
optionally about 0.4% to about 10.4% by weight natural wild berry flavors, citric acid, and organic stevia.

8. The composition of claim 7 comprising:
about 70.6% by weight inulin;
about 7% by weight acerola extract;
about 3% by weight concord grape;
about 3% by weight beet; and
about 2% by weight black current extract having an extract ratio of about 20:1;
about 2% by weight acai;
about 1.5% by weight raspberry extract having an extract ratio of about 20:1;
about 1.5% by weight pomegranate extract, having an extract ratio of about 20:1;
about 1.5% by weight lingonberry extract having an extract ratio of about 20:1;
about 1.5% by weight mangosteen;
about 1% by weight blueberry extract having an extra ratio of about 25:1; and,
optionally about 5.4% by weight natural wild berry flavors, citric acid, and organic stevia.

9. A composition comprising:
about 9% to about 29% by weight maitake mushroom powder;
about 3% to about 23% by weight purple corn powder;
about 0.1% to about 17.1% by weight turmeric powder;
about 3.2% to about 23.2% by weight evaporated cane juice;
about 3.7% to about 23.7% by weight onion;
about 0.1% to about 4.0% by weight basil;
about 0.1% to about 2.8% by weight black pepper;
about 0.1% to about 2.8% by weight coriander;
about 0.1% to about 2.8% by weight oregano;
about 0.1% to about 2.8% by weight rosemary;
about 0.1% to about 2.8% by weight thyme;
about 0.1% to about 0.8% by weight parsley;
about 0.1% to about 0.8% by weight celery seed;
about 0.1% to about 0.4% by weight bay leaf;
about 0.1% to about 19.3% by weight tomato
about 0.1% to about 17.4% by weight garlic;
about 0.1% to about 7.0% by weight yeast;
about 0.1% to about 6.0% by weight salt;
about 0.1% to about 1.0% by weight carrot; and,
about 0.1% to about 1.0% by weight spinach.

10. The composition of claim 9, comprising:
about 19% by weight maitake mushroom powder;
about 13% by weight purple corn powder;
about 7.1% by weight turmeric powder;
about 13.2% by weight evaporated cane juice;
about 13.7% by weight onion;
about 2% by weight basil;
about 1.4% by weight black pepper;
about 1.4% by weight coriander;
about 1.4% by weight oregano;
about 1.4% by weight rosemary;
about 1.4% by weight thyme;
about 0.4% by weight parsley;
about 0.4% by weight celery seed;
about 0.2% by weight bay leaf;
about 9.3% by weight tomato
about 7.4% by weight garlic;
about 3.5% by weight yeast;
about 3% by weight salt;
about 0.5% by weight carrot; and,
about 0.5% by weight spinach.
